(19) United States
(12) Patent Application Publication
(10) Pub. No.: US 2023/0160901 A1
(43) Pub. Date: May 25, 2023

(54) Electrochemiluminescence immunosensor using carbon nanochips, iron oxide and nafion nanocomposite

(71) Applicant: Universiti Brunei Darussalam, Bandar Seri Begawan (BN)

(72) Inventors: Minhaz Uddin AHMED, Bandar Seri Begawan (BN); Juthi ADHIKARI, Bandar Seri Begawan (BN); Mohammad RIZWAN, Bandar Seri Begawan (BN)

(21) Appl. No.: 17/532,871

(22) Filed: Nov. 22, 2021

(51) Int. Cl. G01N 33/577; G01N 21/66; G01N 33/58

(52) U.S. Cl. CPC G01N 33/577; G01N 21/66; G01N 33/583; B82Y 30/00

(57) ABSTRACT

The present application discloses an electrochemiluminescence (ECL) immunosensor. The ECL immunosensor includes an electrode modified by a nanocomposite comprising a mixture of carbon nanochips (CNCs); iron oxide (Fe₃O₄); and nafion (NAF). The electrode is a screen-printed electrode which further is a carbon screen-printed electrode (SPE). The carbon screen-printed electrode (SPE) is a mesoporous carbon screen-printed electrode (SPE). Ru(bpy)₃Cl₂.6H₂O is a luminophore and TPrA is a coreactant of the luminophore.

### TECHNICAL FIELD

The present application relates to detection of exosomes. More particularly, the present application relates to an electrochemiluminescence nanoimmunosensor showcasing a broad linear range to detect CD63 from 100 fg mL⁻¹ to 10 ng mL⁻¹ in clinical samples, and a method of fabricating thereof.

### BACKGROUND

One of the intracellular communications can be through nanosized vesicles, called exosomes. The exosomes are capable of transferring proteins, DNA, micro-RNA, or lipids with or without direct cell to cell. The exosomes can secrete specific proteins and nucleic acids that can cause up and/or down-regulation of physiological processes in respond to local environments, thereby facilitating tumor angiogenesis and metastasis. Such secreted proteins and nucleic acids can further be used as novel biomarkers for the detection and diagnosis of several diseases and ailments. The exosomes can be produced by most eukaryotes. Sources thereof can vary from different cells (e.g., endothelial cells, mast cell, dendritic cells, platelets, neurons, etc.) to various body fluids, such as saliva, blood, amniotic fluid, urine, breast milk, tears, and sweat in human. They also secrete a wide range of proteins including CD63, CD81, CD44, and CD69, to name a few. CD63 is the most extensively studied due to its correlation to several fatal cancers (e.g. breast cancer, ovarian cancer etc.). CD63 protein has four distinct hydrophobic domains, being associated with important cellular functions (i.e. cell development, cell activation and cell motility). In addition, the high level of CD63 protein is linked with cervical cancer, melanoma, and pancreatic cancer as well as others.

There are known conventional methods which can detect and measure the exosomal protein CD63. For example, sandwich ELISA, chemiluminescence, flow cytometry, western blotting, etc. However, the conventional methods require high throughput settings, expert technicians, and are often quite time-consuming from initial sampling until result. Furthermore, the detection of CD63 requires high sensitivity.

Therefore, there exists a need for developing immunosensors which exhibit high sensitivity and less time in spite of cheaper raw materials involved.

### SUMMARY

In a first aspect, the present application discloses a nanocomposite. The nanocomposite includes a mixture of carbon nanochips (CNCs); iron oxide (Fe₃O₄); and nafion (NAF). The nanocomposite includes at least 10 μg mL⁻¹ of the CNCs; and at least 20 μg mL⁻¹ of the Fe₃O₄, and at least 0.25% of the NAF in 1:1 ratio.

In a second aspect, the present application discloses an electrochemiluminescence (ECL) immunosensor. The ECL immunosensor includes an electrode modified by a nanocomposite comprising a mixture of carbon nanochips (CNCs); iron oxide (Fe₃O₄); and nafion (NAF). The electrode is a screen-printed electrode which further is a carbon screen-printed electrode (SPE). The carbon screen-printed electrode (SPE) is a mesoporous carbon screen-printed electrode (SPE). Ru(bpy)₃Cl₂.6H2O is a luminophore and tripropylamine (TPrA) is a coreactant of the luminophore. The immunosensor involves a [Ru(bpy)₃]²⁺/TPrA complex formation between Ru(bpy)₃Cl₂.6H2O and TPrA. The MC-SPE/CNCs/Fe₃O₄/NAF electrode attracts positively charged luminophore via electrostatic interaction. The immunosensor has a CD63 detection range of 100 fg mL⁻¹ to 10 ng mL⁻¹.

In yet another aspect, the present application discloses a method for fabricating an electrochemiluminescence (ECL) immunosensor detecting CD63. The method involves dropping the nanocomposite over bare MC-SPF and drying thereof for at least two hours to form CNC/Fe₃O₄/NAF nanocomposite modified working electrode, followed by spiking anti-CD63 solution over modified-electrode/CNC/Fe₃O₄/NAF. The method further includes incubating the solution overnight at 4° C. to immobilize onto the modified-electrode surface by chemisorption. Thereafter, the MC-SPE/CNC/Fe₃O₄/NAF/anti-CD63 undergoes washing using phosphate-buffered saline (PBS) to remove loosely bound antibody and drying thereof at room temperature (RT), followed by spiking BSA over MC-SPE/CNC/Fe₃O₄/NAF/anti-CD63 as a blocking agent to minimize the non-specific binding and leading to formation of MC-SPE/CNC/Fe₃O₄/NAF/anti-CD63/BSA. The method further involves washing the MC-SPE/CNC/Fe₃O₄/NAF/anti-CD63/BSA using PBS and drying thereof at RT, and finally fabricating the MC-SPE/CNC/Fe₃O₄/NAF/anti-CD63/BSA nanoimmunosensor and storing thereof. The method involves generating light by the immunosensor as an immunocomplex forms between anti-CD63 and CD63 protein over the nanoimmunosensor.

### BRIEF DESCRIPTION OF DRAWINGS

The accompanying figures (FIGS.) illustrate embodiments and serve to explain principles of the disclosed embodiments. It is to be understood, however, that these figures are presented for purposes of illustration only, and not for defining limits of relevant applications.

### FIGURES

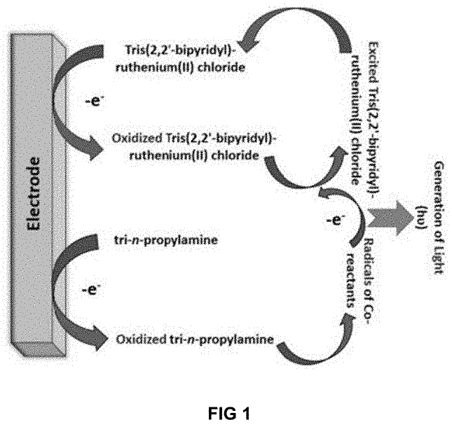

FIG. 1 shows a schematic representing a mechanism of a reaction between a luminophore and a co-reactant over the electrode and transfer of electrons;

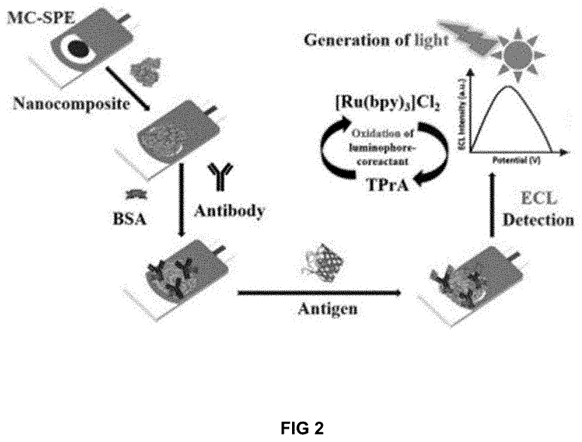

FIG. 2 shows a schematic of a label-free electrochemiluminescence nanoimmunosensor to detect exemplary exosomal protein (CD63);

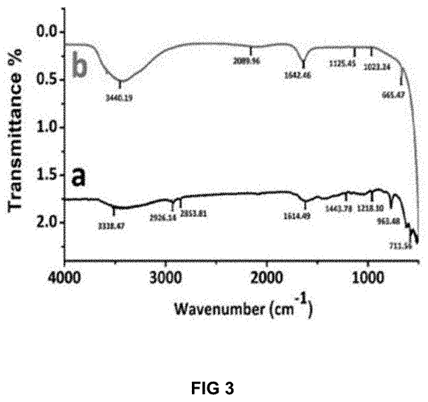

FIG. 3 shows FTIR spectra peak patterns of CNCs and Fe₃O₄ respectively having wavenumber (cm⁻¹) against % of transmittance;

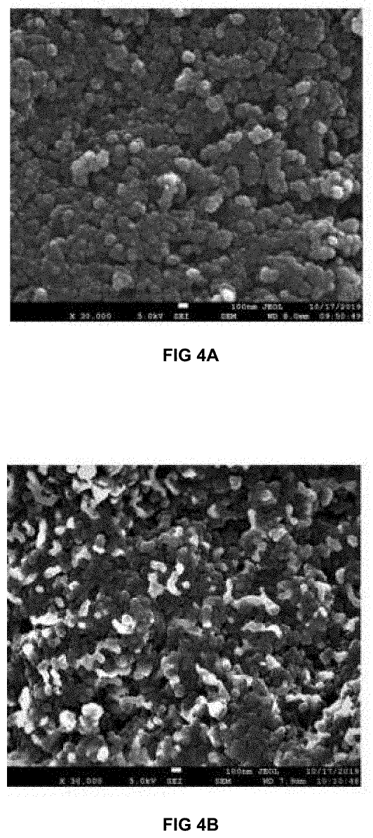

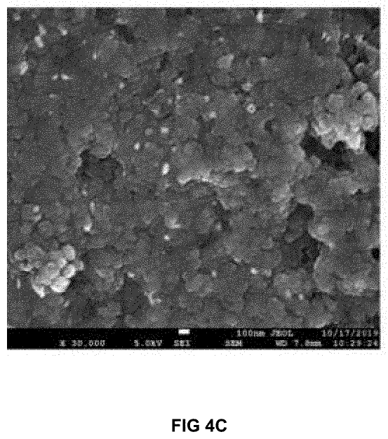

FIG. 4A shows SEM image of MC-SPE;

FIG. 4B shows SEM image of CNCs/MC-SPE;

FIG. 4C shows SEM image of Fe₃O₄/MC-SPE at 30,000×;

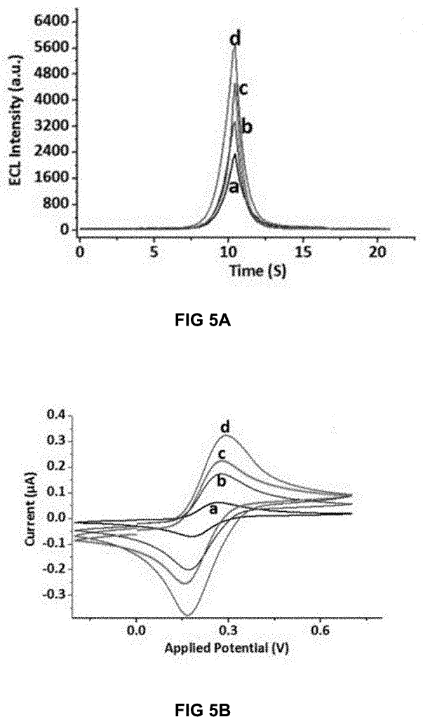

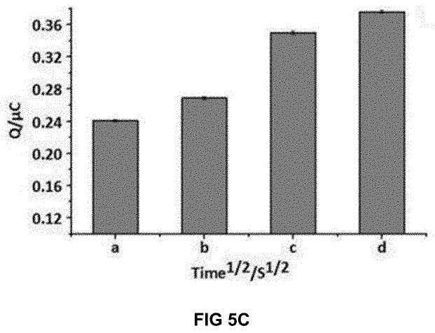

FIG. 5A shows ECL intensity graph of (a) MC-SPE, (b) CNCs/MC-SPE, (c) Fe₃O₄/CNCs/MC-SPE, and (d) NAF/Fe₃O₄/CNCs/MC-SPE;

FIG. 5B shows electrochemical graph of (a) MC-SPE, (b) CNCs/MC-SPE, (c) Fe₃O₄/MC-SPE, (d) Fe₃O₄/CNCs/MC-SPE;

FIG. 5C shows CC bar graph of (a) MC-SPE, (b) CNCs/MC-SPE, (c) Fe₃O₄/MC-SPE, and (d) Fe₃O₄/CNCs/MC-SPE;

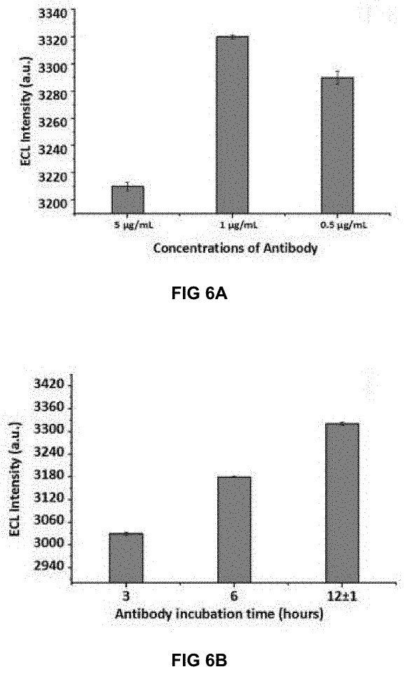

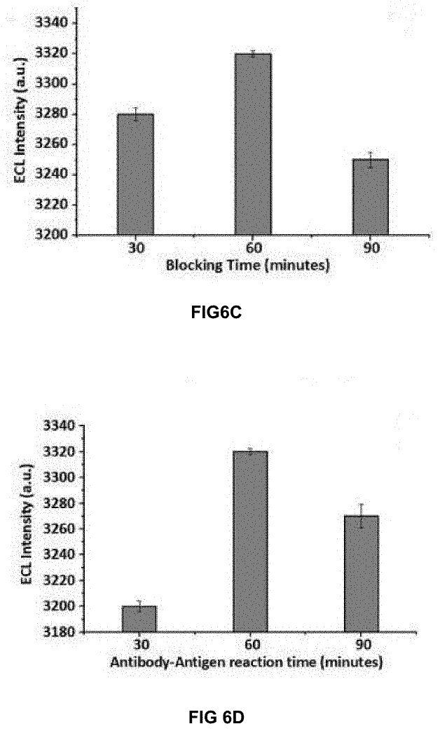

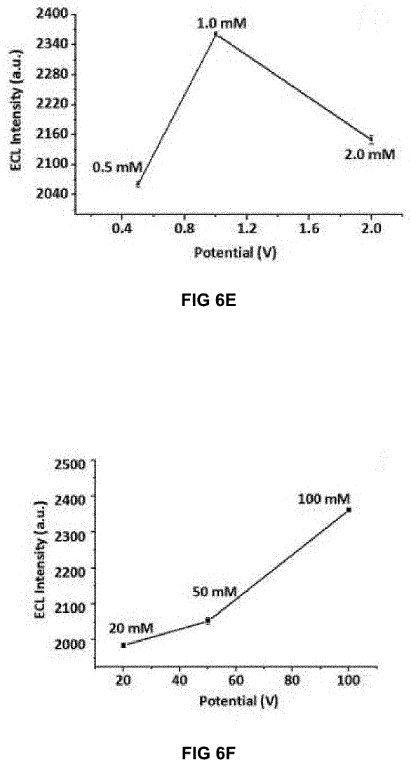

FIG. 6A shows a bar graph depicting optimization of anti-CD63 working concentration (5 μg mL⁻¹, 1 μg mL⁻¹, and 0.5 μg mL⁻¹) of the anti-CD63, spiked on the MC-SPE/CNCs/Fe₃O₄/NAF immunosensor with 100 pg mL⁻¹ CD63;

FIG. 6B shows a bar graph depicting optimization of anti-CD63 (1 μg mL⁻¹) incubation time on MC-SPE/CNCs/Fe₃O₄/NAF platform with 100 pg mL⁻¹ CD63;

FIG. 6C shows an optimization of blocking time by 0.1% BSA in 0.1% NaN₃ prepared in 10 mM PBS, pH 7.4 spiked on MC-SPE/CNCs/Fe₃O₄/NAF/anti-CD63 for 30, 60 and 90 min with 100 pg mL⁻¹ CD63;

FIG. 6D shows a bar graph showing anti-CD63 and CD63 protein reaction being carried out for 30, 60 and 90 min with 100 pg mL⁻¹ CD63 on MC-S PE/CNCs/Fe₃O₄/NAF/anti-CD63/BSA;

FIG. 6E shows a graph depicting optimization of [Ru(bpy)₃]²⁺ working concentration;

FIG. 6F shows a graph depicting optimization of TPrA working concentrations;

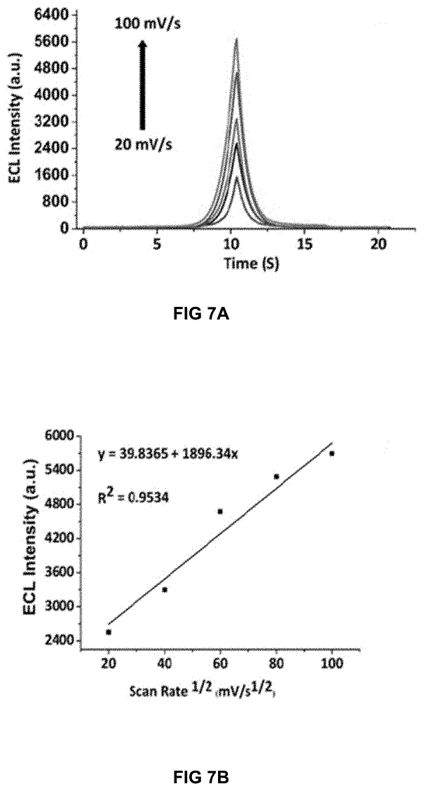

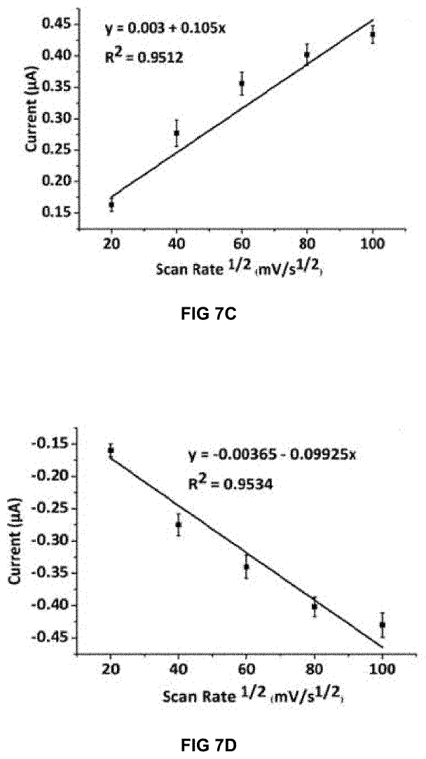

FIG. 7A shows ECL intensity of the fabricated immunosensor from 20 mVs⁻¹ to 100 mVs⁻¹;

FIG. 7B shows a graph depicting dependence of oxidation peak currents on the square root of the scan rates using 1:100 [Ru(bpy)₃]Cl₂-TPrA;

FIG. 7C shows a graph depicting dependence of oxidation peak currents on the square root of the scan rates;

FIG. 7D shows a graph representing dependence of reduction peak currents on the square root of the scan rates utilizing 5 mM [Fe(CN)₆]³⁻/[Fe(CN)₆]⁴⁻ redox probe;

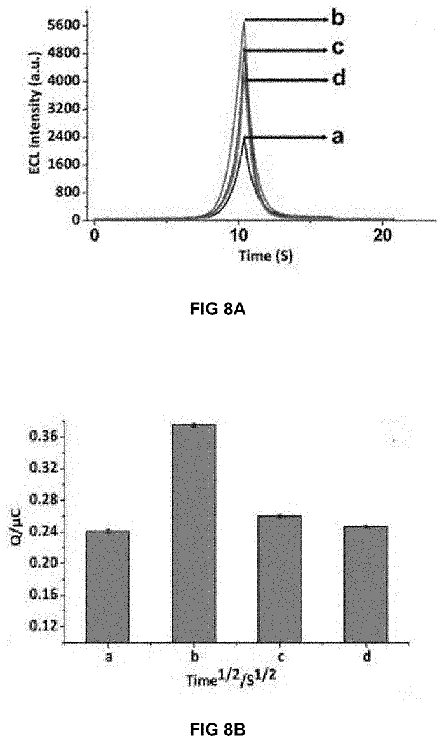

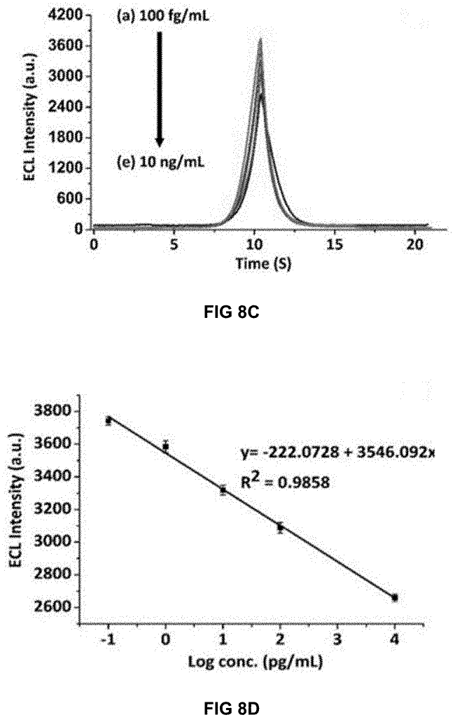

FIG. 8A shows an ECL intensity curve of: (a) MC-SPE, (b) MC-SPE/CNCs/Fe₃O₄/NAF, (c) MC-SPE/CNCs/Fe₃O₄/NAF/anti-CD63, (d) MC-SPE/CNCs/Fe₃O₄/NAF/anti-CD63/BSA/CD63 measured in luminophore solution, CSE=100 mVs⁻¹ using 1:100 [Ru(bpy)₃]Cl₂-TPrA solution containing 10 mM PBS, pH 7.4;

FIG. 8B shows a CC bar of: (a) MC-SPE, (b) MC-SPE/CNCs/Fe₃O₄/NAF, (c) MC-SPE/CNCs/Fe₃O₄/NAF/anti-CD63, (d) MC-SPE/CNCs/Fe₃O₄/NAF/anti-CD63/BSA/CD63, measured in redox probe (5 mM [Fe(CN)₆]³⁻/[Fe(CN)₆]⁴⁻) solution, CSE=100 mVs⁻¹;

FIG. 8C shows ECL dose response curves representing different concentrations of CD63 from 10 ng mL⁻¹ to 100 fg mL⁻¹: (a) 100 fg mL⁻¹, (b) 100 pg mL⁻¹, (c) 10 pg mL⁻¹, (d) 1 pg mL⁻¹, (e) 10 ng mL⁻¹;

FIG. 8D shows a calibration plot of the CD63 nanoimmunosensors towards different concentrations from 10 ng mL⁻¹ to 100 fg mL⁻¹ of CD63 (n=3);

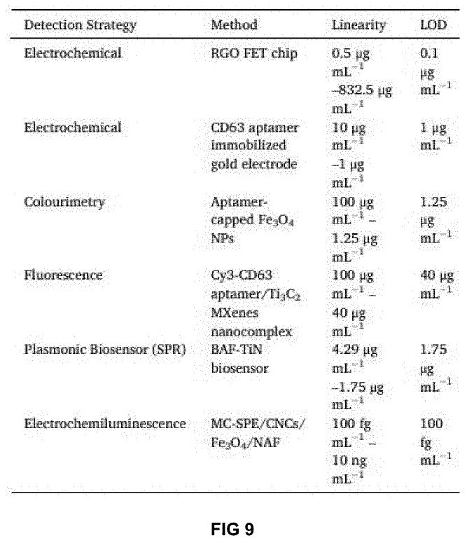

FIG. 9 shows a comparison of different sensing strategies with designed immunosensor for CD63 detection;

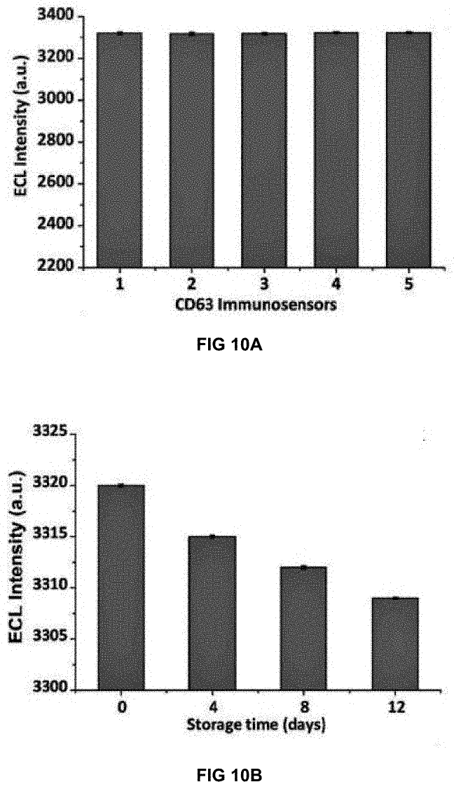

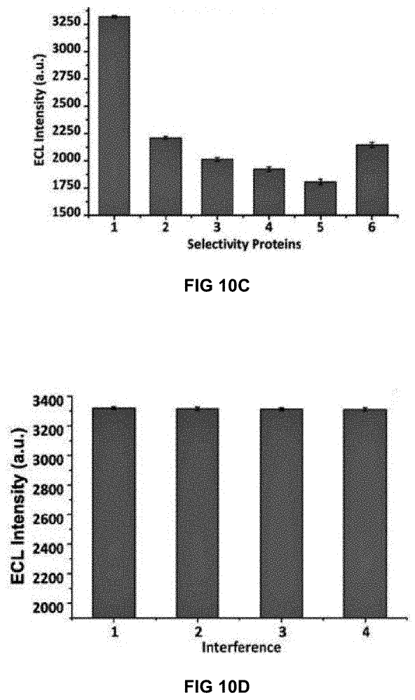

FIG. 10A shows a record of signals of five fabricated MC-SPE/CNCs/Fe₃O₄/NAF/anti-CD63/BSA nanoimmunosensors tested with 100 pg mL⁻¹ CD63;

FIG. 10B shows bar graphs representing storage of MC-SPE/CNCs/Fe₃O₄/NAF/anti-CD63/BSA nanoimmunosensor at 4° C. and testing thereof with 100 pg mL⁻¹ CD63 at different five days interval;

FIG. 10C shows a bar graph representing record of signals of CD63 nanoimmunosensor tested against other analytes present in serum;

FIG. 10D shows a bar graph representing record of signals of CD63 nanoimmunosensor tested against 100 pg mL⁻¹ of CD63 in combination with 100 pg mL⁻¹ of other analytes present in serum;

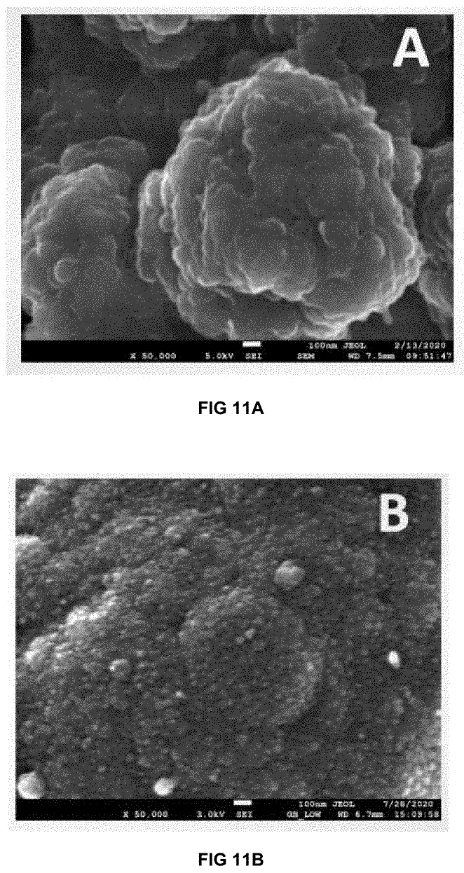

FIG. 11A shows a SEM image of MC-SPE/CNCs/Fe₃O₄/NAF/antiCD63/BSA;

FIG. 11B shows a SEM image of MC-SPE/CNCs/Fe₃O₄/NAF/anti-CD63/BSA/CD63;

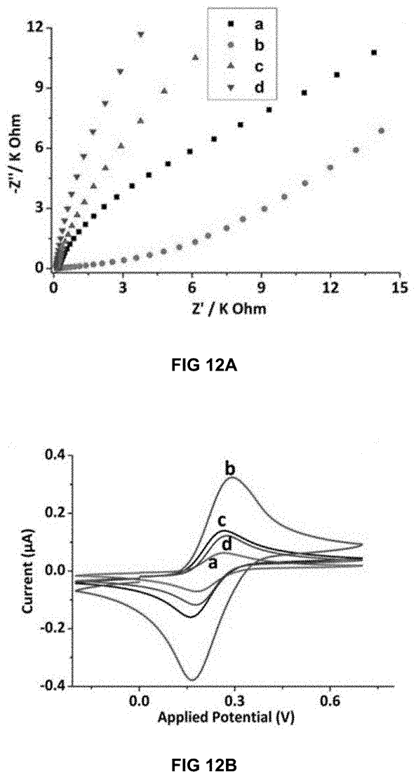

FIG. 12A shows an electrochemical impedance spectroscopy (EIS) analysis through Nyquist plots of impedance spectra for (a) bare MC-SPE, (b) MC-SPE/CNCs/Fe₃O₄, (c) with MC-SPE/CNCs/Fe₃O₄/anti-CD63, (d) with MC-SPE/CNCs/Fe₃O₄/antiCD63/BSA/CD63;

FIG. 12B shows Cyclic Voltammetry curves of (a) bare, (b) MC-SPE/CNCs/Fe₃O₄/NAF nanocomposite, (c) with MC-SPE/CNCs/Fe₃O₄/NAF/anti-CD63, and (d) with MCSPE/CNCs/Fe₃O₄/NAF/anti-CD63/BSA/CD63 using 5 mM [Fe(CN)₆]³⁻/[Fe(CN)6]⁴⁻ redox probe (n=3);

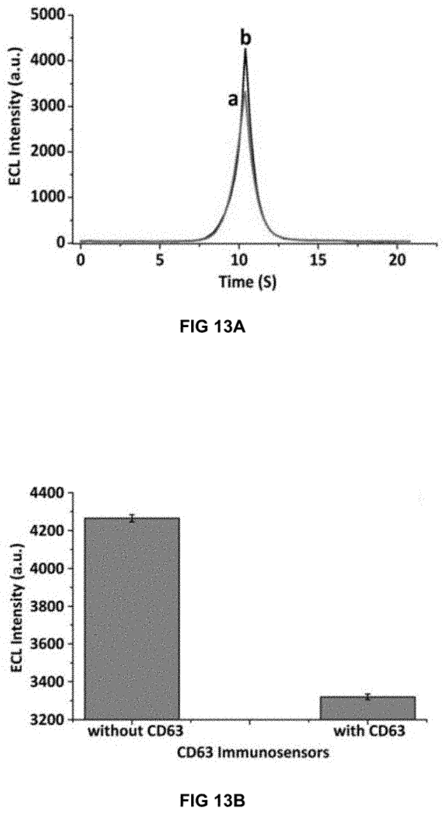

FIG. 13A shows a curve of ECL intensity of the fabricated CD63 nanoimmunosensor (MC-SPE/CNCs/Fe₃O₄/NAF/anti-CD63/BSA): (a) with CD63, and (b) without CD63;

FIG. 13B shows a bar graph depicting record of the ECL signal on the sensor with 100 pg mL⁻¹ CD63 and without CD63 (n=3).

### DETAILED DESCRIPTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

ECL is an electrochemical process between luminophore and co-reactant which produces light when a specific voltage is applied. Most commonly, the co-reactant ECL pathway is utilized for analytical detection methods. Other advantages of ECL includes the absence of background signal, flexibility thereof and suitability to many different electrode materials, sizes and dimensions, reaction position, reaction time, as well as control over applied potential and currents. There are different types of electrodes, for example screen-printed electrodes (SPE), glassy carbon electrodes (GCE), or disposable electrode printed chips (DEP-chips) which can be used in designing ECL based sensors establishing ECL as both a diverse and versatile analytical detection method. The applied voltage causes the luminophore to be oxidized, donating electrons at the electrode surface. However, as the thickness of the antibody-antigen increases over the electrode, the interactions between luminophore and electrode decrease. Further, the ECL intensity may increase and decrease depending on the net charge of the implied biomolecules on the working electrode surface.

Therefore, the present application involves utilization of ECL immunosensor for detecting CD63 by modifying existing screen-printed electrodes (SPE). In the embodiment, The ECL immunosensor includes an electrode modified by a nanocomposite. The nanocomposite includes a mixture of carbon nanochips (CNCs); iron oxide (Fe₃O₄); and nafion (NAF). In the embodiment, the nanocomposite includes at least 10 μg mL⁻¹ of the CNCs; and at least 20 μg mL⁻¹ of the Fe₃O₄, and at least 0.25% of the NAF in 1:1 ratio. The CNCs are two-dimensional (2D) carbon sheets resembling a similar structure of carbon nanotubes having exceptional mechanical and electrical conductivity. For instance, the CNCs reduce the rate of biofouling during analysis, and promote electron transfer on the working electrode. The Fe₃O₄ is well known for versatility thereof for the enhancement of sensor performance.

For the ECL immunosensor, the electrode is a screen-printed electrode which further is a carbon screen-printed electrode (SPE). The carbon screen-printed electrode (SPE) is a mesoporous carbon screen-printed electrode (SPE). Ru(bpy)₃Cl₂.6H2O is a luminophore and TPrA is a coreactant of the luminophore. The immunosensor involves a [Ru(bpy)₃]²⁺/TPrA complex formation between Ru(bpy)₃Cl₂.6H2O and TPrA. The MC-SPE/CNCs/Fe₃O₄/NAF electrode attracts positively charged luminophore via electrostatic interaction. The immunosensor has a CD63 detection range of 100 fg mL⁻¹ to 10 ng mL⁻¹.

The NAF may be added to the CNCs/Fe₃O₄ composite to achieve a unique negatively charged engineered nanocomposite (CNCs/Fe₃O₄/NAF). The main function of the nanocomposite is to facilitate high electron transfer during the ECL reaction. Therefore, CNCs/Fe₃O₄/NAF nanocomposite attracts the positively charged luminophore (tris(2,2′-bipyridyl) dichlororuthenium (II) hexahydrate); Ru(bpy)₃]Cl₂.6H2O, over the MCSPE/CNCs/Fe₃O₄/NAF electrode by attractive electrostatic interaction, enabling more electron transfer between the modified-electrode surface (MC-SPE/CNCs/Fe₃O₄/NAF) and [Ru(bpy)₃]²⁺/TPrA complex via redox reaction providing high ECL signal. The mechanism of the reaction between the luminophore (Ru(bpy)₃]Cl₂.6H₂O) and the co-reactant (TPrA) and transfer of electrons is shown in FIG. 1. The fabricated nanoimmunosensor highly sensitive, efficient, and stable.

The fabricated immunosensor (MC-SPE/CNC/Fe₃O₄/NAF/anti-CD63/BSA) showcased notable stability and reproducibility in detecting target protein, CD63 and exhibited a wide linear range 100 fg mL⁻¹ to 10 ng mL⁻¹ and a low detection limit of 100 fg mL⁻¹ for the detection of CD63.

Materials and Methods

Rabbit monoclonal antibody CD63 and exosomal protein CD63, CD81, CD69, bovine serum albumin (BSA), carcino-embryogenic antigen (CEA), alpha fetoprotein (AFP), haptoglobin (Hp), sodium azide, potassium chloride, potassium ferrocyanide, potassium ferricyanide, tris (2,2′-bipyridyl) dichlororuthenium(II) hexahydrate, tripropylamine, tris-disodium phosphate, and monosodium phosphate, nanocomposite binding agent, carbon nano chips (CNCs), 5% NAF solution were purchased from Sigma-Aldrich (USA). Iron oxide (Fe₃O₄) nanoparticles were procured from US Research Nanomaterials, Inc. (Houston, USA). All solutions were prepared using freshly obtained Milli-Q water (deionized with specific resistance ˜18 M cm⁻¹). All the experiments were performed at the room temperature (RT) (21±0.5° C.).

All the ECL measurements were performed utilizing an MPI-A capillary electrophoresis electrochemiluminescence analyzer system, purchased from Xi'anYima Opto-Electrical Technology Co., Ltd. (China). A handmade ECL working cell (height 5 cm, width 1.5 cm) was utilized to detect the light generated from the reactions between ECL probe and the electrode surface. The ECL cell was placed on top of a photomultiplier tube (PMT) which was connected to the MPI-A software to analyze ECL intensity. The fabricated sensor was immersed in the ECL cell containing luminophore-coreactant mixture ([Ru(bpy)₃]Cl₂-TPrA) and placed on the PMT to conduct ECL measurement. The electrochemical layer-by-layer characterization studies cyclic voltammetry (CV), chronocoulometry (CC), and electrochemical impedance spectroscopy (EIS) were carried out using Autolab PGSTAT101 III potentiostat/galvanostat (Metrohm, The Netherlands) connected to a Nova software version 1.10. The disposable screen-printed electrodes were purchased from DropSens (Spain), where the working electrode was modified with mesoporous carbon, reference electrode with silver, and the counter electrode with carbon. The diameter of the mesoporous carbon modified working electrode was 4 mm. The overall dimensions of these non-reusable ceramic electrodes are (L33×H0.5×W10) mm. The surface topographical study was done by using field-emission electron microscopy (FE-SEM) JEOL, JSM-7610F (Japan). Fourier transform-infrared (FTIR) spectroscopy (Shimadzu, Japan) was used for the analysis of nanocomposite. All experiments were performed at constant room temperature (21±0.5° C.) and atmospheric pressure in an air-conditioned laboratory. All experimental data are an average of three replicates achieved from three different fabricated sensors maintaining similar optimal condition.

The preparation of the selected CNCs/Fe₃O₄/NAF nanocomposite was done in-house at 21±0.5° C. The CNCs and Fe₃O₄ were prepared in two separate small glass vials by dissolving in dH₂O and were ultra-sonicated for 3.5 h for uniform dispersion. Thereafter, the sonicated nanoparticles were gradually diluted to achieve the optimum concentrations (10 mL⁻¹ for CNCs and 20 μg mL⁻¹ for Fe₃O₄). In the meantime, 0.25% NAF was also prepared by serially diluting it using double dH₂O from the main stock. Finally, the synthesis of the final nanocomposite was performed by mixing 10 μg mL⁻¹ for CNCs, 20 μg mL⁻¹ for Fe₃O₄, and 0.25% NAF at 1:1 ratio and stirring it at a magnetic stirrer for 6 h. The resulting nanocomposite mixture was stored at 4° C. and was ultra-sonicated for 60 min before each use.

The present application discloses a flowchart depicting a method for fabricating the ECL immunosensor as shown in FIG. 2. Firstly, CNC/Fe₃O₄/NAF nanocomposite was dropped over bare MCSPE and dried for two hours to form CNC/Fe₃O₄/NAF nanocomposite modified working electrode MC-SPE (MC-SPE/CNC/Fe₃O₄/NAF). Then, 10 μL of the anti-CD63 solution (1 μg mL⁻¹) was spiked over MC-SPE/CNC/Fe₃O₄/NAF and incubated overnight at 4° C. to immobilize onto the electrode surface by chemisorption. Next, MC-SPE/CNC/Fe₃O₄/NAF/antiCD63 was washed using 10 mM PBS (pH 7.4) to remove loosely bounded antibody and dried at RT. Following washing and drying, 0.1% BSA was spiked over MC-SPE/CNC/Fe₃O₄/NAF/anti-CD63 as the blocking agent to minimize the non-specific binding and formation of MC-SPE/CNC/Fe₃O₄/NAF/anti-CD63/BSA. Later, MC-SPE/CNC/Fe₃O₄/NAF/antiCD63/BSA was washed using PBS (pH 7.4) and dried at RT. Finally, the fabricated MC-SPE/CNC/Fe₃O₄/NAF/anti-CD63/BSA nanoimmunosensor was stored at 4° C. until further use.

The ECL detection of CD63 was accomplished by applying different concentrations of CD63 on MC-SPE/CNC/Fe₃O₄/NAF/anti-CD63/BSA nanoimmunosensor. To obtain the ECL measurements for each of the CD63 concentrations, 10 μL of CD63 was incubated on nanoimmunosenor for 60 min (at RT 21±0.5° C.) followed by washing (with 10 mM PBS, pH 7.4) and drying. The ECL detection was performed by pre-making the ECL probe mixture. The total volume of the ECL probe was 4 mL containing 1 mL of [Ru(bpy)₃]²⁺ (1 mM) and TPrA (100 mM) each, and 2 mL of 10 mM PBS having pH 7.4. The glass cell was entirely covered with aluminum foil paper, only exposing the bottom section (diameter 1.5 cm) to authorize the diffusion of light over the PMT. The ECL cell was kept on top of the PMT which was in a lightproof black box to ensure the maximum performance of the ECL analyzer without the disturbance of external light source. All ECL measurements were performed using primary potential of 0.2 V, end potential of 1.25 V and the lowest potential as −0.2 V. The selected scan rate was 100 mVs⁻¹ with an amplifying series of 3, sensitivity 1×10⁻⁶ and PMT potential of 800 V. Maximum ECL intensity was obtained at ˜10 s after starting each cycle. The error bars signify the relative standard deviations of at least three replicates (n=3) for all experiments.

The composition of CNCs/Fe₃O₄ nanocomposite was analyzed using FTIR. 2 mg of the nanocomposite sample was mixed with 200 mg KBr to form a pellet of fine consistency. FIG. 3 represents the prominent peak patterns of (a) CNCs and (b) Fe₃O₄ respectively having a wavenumber range between 800 and 4000 cm⁻¹ and a transmittance percentage (%) from 0 to 2.5. The major peak differences have been observed between 700 cm⁻¹ to 1600 cm⁻¹ regions and the percentage of peak transmittance starts from 0.2% Fe₃O₄ and 1.8% for CNCs. For CNCs, peak at 1614.49 cm⁻¹ region shows the C═C stretching whereas spectra at 2926.14 cm⁻¹ and 2853.81 cm⁻¹ exhibited the —CH₂ alignment respectively]. In the case of Fe₃O₄, the peak at 3440.19 cm⁻¹ regions signifies the O—H stretching group. Additionally, 1642.46 cm⁻¹, 1023.48 cm⁻¹ denote the surface hydroxyl group and nitrate stretching vibrations whereas peaks at 956.41 cm⁻¹ represent the F—O alignment. Thus, the FTIR spectrum successfully confirms the presence of CNCs and Fe₃O₄ within the nanocomposite.

In order to confirm the structure of the nanocomposite layer on MCSPE, each nanoparticle was tested using FE-SEM. Nanoparticles were individually incubated on MC-SPE as shown in FIG. 4A and analyzed using FE-SEM. CNCs exhibited fibrous-small thread-like structures as illustrated in FIG. 4B, whereas iron oxide (Fe₃O₄) showed small spherical like orientations as shown in FIG. 4C. In addition, FIG. 11A and FIG. 11B display the nanocomposite layer incubated with antibody and antigen respectively highlight extremely distinct features. For example, large assemblies of antibody and visibility of BSA as shown in FIG. 11A. Whilst, in the case of FIG. 11B, the big alignments were covered by antigen layer exhibiting a smooth surface possibly due to the immunocomplex formation between anti-CD63 and CD63 protein over the nanoimmunosensor.

Further, characterization of the CNCs/Fe₃O₄/NAF nanocomposite was performed by EC and ECL methods. The ECL produced from each layer of the nanocomposite modified on the surface of the MC-SPE was recorded to investigate the ECL enhancement observed due to the addition of the nanoparticles. The recorded ECL signal for nanoparticle and nanocomposite was compared and plotted against bare MC-SPE in FIG. 5A. There was a large increase in the ECL intensity of CNCs/Fe₃O₄/NAF nanocomposite modified MC-SPE as shown in curve d of FIG. 5A as compared to that of the bare MC-SPE shown in curve a of FIG. 5A.

Following ECL characterization of the nanocomposite layer, the nanocomposite was subjected to electrochemical characterization, where the nanoparticle and their composite was characterized applying CV based on their response against a constant set of potential. Similar trend appeared for each layer of the nanoparticles and the composite thereof on electrochemical analysis when compared to ECL. After incubating the working electrode with CNCs, there was a significant rise in electrochemical response as shown in curve b of FIG. 5B in comparison to that of the bare MC-SPE as shown in curve a of FIG. 5B. After the addition of Fe₃O₄, the electrochemical signal was further enhanced due to the highly conductive properties of the iron oxide nanoparticles as shown in curve c of FIG. 5B. And finally, the addition of the nanocomposite (CNCs/Fe₃O₄) on MC-SPE, showcased the maximum electrochemical current as shown in curve d of FIG. 5B.

Thereafter, the CC study was conducted for the MC-SPE as shown in bar a of FIG. 5C, CNCs/MC-SPE shown in bar b of FIG. 5C, Fe₃O₄/CNCs/MC-SPE shown in bar c of FIG. 5C, and Fe₃O₄/CNCs/MC-SPE shown in bar d of FIG. 5C. Such results demonstrate a gradual increase in the electrostatic charge in correlation to the ECL and electrochemical study.

The optimization of antibody (anti-CD63), was accomplished by examining three different concentrations of anti-CD63 (5 μg mL⁻¹, 1 μg mL⁻¹ and 0.5 μg mL⁻¹) on the MC-SPE/CNCs/Fe₃O₄/NAF platform at 21±0.5° C. using 100 pg mL⁻¹ CD63 as illustrated in FIG. 6A. Subsequently, 1 μg mL⁻¹ was chosen as the optimum working concentration to develop MC-SPE/CNCs/Fe₃O₄/NAF/anti-CD63 nanosensor to capture out target antigen (CD63). Herein, blocking was performed for 60 min, and immune-complex (antibody-antigen) formation time was 1 h at 21±0.5° C. as shown in FIG. 6B. Following that, the incubation time for CD63 antibody was selected by optimizing three distinct periods (3, 6 and 12±1 h) where maximum and stable CV response was measured for 12±1 h incubation of the antibody using 100 pg mL⁻¹ CD63. Henceforth, 12±1 h was chosen as the optimum incubation time for anti-CD63 (FIG. 6B). Therefore, blocking was performed for 60 min, and the antigen-antibody reaction was carried out for 1 h at 21±0.5° C. in 10 mM PBS, pH 7.4 (n=3).

The selection of the incubation time of blocking agent, BSA was achieved by preparing 0.1% BSA in 0.1% NaN₃ in 10 mM PBS (pH 7.4) and 30, 60 and 90 min incubation times were verified to mitigate the nonspecific binding at the surface of MC-SPE/CNCs/Fe₃O₄/NAF/antiCD63/BSA immunosensor using 100 pg mL⁻¹ CD63. The highest ECL peak was recorded at 60 min as depicted in FIG. 6C, so 1 h was selected as the optimum blocking time for the immunosensor. Thereafter, to optimize the immunocomplex formation time between anti-CD63 and CD63 protein (100 pg mL⁻¹), three different time (30, 60, and 90 min) were tested on MC-SPE/CNCs/Fe₃O₄/NAF/anti-CD63/BSA nanosensor, where 60 min showcased maximum and repeatable ECL intensity. Henceforth, 1 h was selected as the optimum antibody-antigen reaction time between anti-CD63 and CD63 protein as shown in FIG. 6D at 21±0.5° C. in 10 mM PBS, pH 7.4 (n=3).

For the optimization of the [Ru(bpy)₃]²⁺, different concentrations (0.5 mM, 1.0 mM and 2.0 mM), of [Ru(bpy)₃]²⁺ were tested and 1 mM [Ru(bpy)₃]²⁺ was found with decent stability and reproducibility. Henceforth, 1 mM [Ru(bpy)₃]²⁺ was chosen as the optimum concentration to combine with TPrA as clearly depicted in FIG. 6E. Simultaneously, the working concentration of TPrA was selected after trying three different concentrations (20 mM, 50 mM and 100 mM), where 100 mM of TPrA was found to have superior stability as illustrated in FIG. 6F. The working ratio of [Ru(bpy)₃]²⁺ and TPrA was chosen as 1:100.

Both ECL and electrochemical scan rates study was performed to demonstrates the electrochemical diffusion over nanoimmunosensors. FIG. 7A shows the increase in ECL intensity as related to the increase in scan rates, and the maximum ECL signal was recorded at a scan rate of 100 mVs⁻¹. Whereas, FIG. 7B showcase the oxidation peaks vs square root of scan rates exhibiting a linear relationship between the tested scan rates. Likewise, the diffusion kinetics study was performed using [Fe(CN)₆]³⁻/[Fe(CN)₆]⁴⁻ on the nanoimmunosensor surface applying electrochemical diffusion kinetic, at scan rates 20 mVs⁻¹ to 100 mVs⁻¹. The linear relationship of the oxidation-reduction peaks and the square root of the scan rate was demonstrated by the nanoimmunosensors respectively in FIGS. 7C and 7D. Further, heights of the peaks increase with the increasing scan rates show the dependency on electrochemical diffusion.

For the layer-by-layer ECL characterization of the MC-SPE/CNCs/Fe₃O₄/NAF/anti-CD63/BSA nanoimmunosensor, each layer was individually incubated, and data were analyzed. Firstly, the ECL intensity for the bare MC-SPE was recorded as shown in curve a of FIG. 8A. Thereafter, the ECL intensity was measured after incubating with CNCs/Fe₃O₄/NAF nanocomposite, which exhibited a significant increase in ECL peak signal compared to the bare electrode as shown in curve b of FIG. 8A. The maximum rise in the ECL signal was due to the high electrochemical conductivity of the nanoparticles (CNCs, Fe₃O₄) used in preparing the nanocomposite specifically for this working electrode. Then the anti-CD63 was immobilized on the MC-SPE/CNCs/Fe₃O₄/NAF surface to develop MC-SPE/CNCs/Fe₃O₄/NAF/anti-CD6, thereby showing a slight decrease in the ECL signal due to quenching of tris (2,2′-bipyridyl) dichlororuthenium(II) activity over MC-SPE/CNCs/Fe₃O₄/NAF/anti-CD63 because of the formed insulating layer of anti-CD63, showing the successful adsorption of the antibody layer in curve c of FIG. 8A. Finally, MC-SPE/CNCs/Fe₃O₄/NAF/anti-CD63 nanosensor was incubated with 10 μL of 0.1% blocking agent (BSA) to inhibit non-specific binding sites the electrode surface. ECL intensity further decreased due to further quenching of tris (2,2′bipyridyl) dichlororuthenium(II) activity over MC-SPE/CNCs/Fe₃O₄/NAF/anti-CD63/BSA because of the formation of another insulating layer of BSA, portraying the adsorption of the BSA to form MC-SPE/CNCs/Fe₃O₄/NAF/anti-CD63/BSA nanoimmunosensor as shown in curve d of FIG. 8A. Further, surface charge was evaluated for each layer of the immunosensor using CC as shown in FIG. 8B. The surface charge layer of the nanoimmunosensors shows a correlation with the ECL intensity. In addition, layer-by-layer characterization studies were performed via EIS and CV, in FIGS. 12A and 12B, respectively.

To evaluate the analytical performance of the immunosensor, the MC-SPE/CNCs/Fe₃O₄/NAF/anti-CD63/BSA platform was incubated with different concentrations (100 fg mL⁻¹ to 10 ng mL⁻¹) of CD63 as shown in FIG. 8C. A calibration plot was plotted using the log concentrations of the CD63 antigen, ranging from 100 fg mL⁻¹ to 10 ng mL⁻¹ against ECL intensity under optimum conditions (at 21±0.5° C. in an air-conditioned laboratory, at 10 mM PBS pH 7.4 using 100 mM [Ru(bpy)₃]Cl₂ 1 mM TPrA. The linearity graph of FIG. 8D shows a negative linear relationship between the aforementioned concentration range (100 fg mL⁻¹ to 10 ng mL⁻¹; R2=0.9858) and ECL intensity. Hence, as the concentration of CD63 increases from 100 fg mL⁻¹ to 10 ngmL⁻¹ (FIG. 8C) over SPE/CNCs/Fe₃O₄/NAF/anti-CD63/BSA nanoimmunosensor quenching of tris (2,2′-bipyridyl) dichlororuthenium(II) activity over SPE/CNCs/Fe₃O₄/NAF/anti-CD63/BSA increases due to formed thick insulating layer of CD63 protein. Further, CD63 protein is slightly positively charged in the working pH (7.4) having a pI of 8.1. Consequently, with increasing CD63 concentration, the ECL intensity faces a decreasing trend and vice versa as shown in FIG. 8D. The limit of detection (LOD) of the immunosensor was found to be 100 fg mL⁻¹, which can be repeatedly detected by the CD63 specific immunosensor. The low LOD is due to the enhanced ECL activity and conductivity generated from the CNCs/Fe₃O₄/NAF nanocomposite. Besides, the large surface area of the MC-SPE with the enhanced electrochemical active area and enhanced electronic transfer properties also help in quick electron transfer, and thus improve the overall performance of the fabricated immunosensor. A comparison of different sensing strategies with designed immunosensor for CD63 detection has been shown in FIG. 9.

Further, the label-free MC-SPE/CNCs/Fe₃O₄/NAF/anti-CD63/BSA was authenticated for its ability to detect 100 pg mL⁻¹ CD63, which was recorded as ECL intensity plotted against time in FIG. 13A and shown by bars in FIG. 13B. The analytical efficiency of the proposed sensor was verified by the relative standard deviation (RSD %), less than 5%. The target protein (CD63) has an isoelectric point (pI) of 8.1 carrying positive charge at the working pH 7.4, repulsing the positively charged luminophore (Ru(bpy)₃]Cl₂) and co-reactant (TPrA). Consequently, the diffusion of Ru(bpy)₃]Cl₂-TPrA onto the immunocomplex was hindered with the increasing CD63 concentration resulting in a declined ECL signal. For additional validation that the ECL intensity is dependent on the Ru(bpy)₃]Cl₂-TPrA diffusion process. ECL study was performed which showed that in the presence of the target protein, the total ECL intensity determined was condensed demonstrating that less [Ru(bpy)₃]Cl₂ was dispersed towards CD63 antigen.

The reproducibility of the developed MC-SPE/CNCs/Fe₃O₄/NAF/anti-CD63/BSA nanoimmunosensor was examined by fabricating five electrodes at different times and later evaluating their respective signals. The concentrations of CD63 used for testing each electrode fabrication was 100 pg mL⁻¹ as shown in FIG. 10A. The ECL immunosensor shows significant reproducibility with a potential to clinical sample analysis to detect CD63 protein. Further, the stability of the fabricated immunosensor was calculated by storing four electrodes up to twelve days, having a time interval of four days as illustrated in FIG. 10B. The electrodes showcased a gradual descending trend of signals upon storing at 4° C., demonstrating long term stability patterns. Thereafter, the selectivity of the MC-SPE/CNCs/Fe₃O₄/NAF/anti-CD63/BSA nanosensor was investigated. Two different types of exosomal proteins (CD69, CD81: concentration 100 pgmL⁻¹) were tested on fabricated MC-SPE/CNCs/Fe₃O₄/NAF/anti-CD63/BSA nanoimmunosensors. Thereafter, results were compared with that of 100 pgmL⁻¹ of CD63 as shown in FIG. 10C. Moreover, three more serum proteins CEA, AFP and Hp, were also tested for further confirmation of the selectivity performance of the ECL immunosensor as shown in FIG. 10C. The results indicate that the MC-SPE/CNCs/Fe₃O₄/NAF/anti-CD63/BSA immunosensor was extremely specific to the target protein CD63. Finally, the ECL immunosensor underwent testing with the mixture of non-target proteins for the real clinical sample analysis, and the signal was compared with that of CD63 (target protein) applying the same working volume and concentration as shown in FIG. 10D. All the aforementioned data reassure the efficiency and potentiality of the fabricated MC-SPE/CNCs/Fe₃O₄/NAF/anti-CD63/BSA nanoimmunosensor to detect CD63 protein to be used as a point-of care-testing device.

Further elaborating analysis of SEM images shown in FIGS. 11A and 11B. For the EIS measurements, 5 mM K₃[Fe(CN)₆]/K₄[Fe(CN)₆] mixture was used as redox probe with the frequency range between 100 kHz to 0.1 Hz, ac amplitude 10 mV and the number of frequency of 50 Hz. The pattern of the EIS plots were semi-circles along with some straight-line portions which represented the charge transfer resistance (Rct) and diffusion-controlled processes respectively. In the Nyquist equation, the charge transfer resistance (Rct) is related to the diameter of the semi-circle.

As shown in FIG. 12A, the EIS pattern for all the layers are presented individually, where (a) was the plot for the bare MC-SPE electrode, (b) was the semi-circle of CNCs/Fe₃O₄/NAF nanocomposite modified-electrode, (c) was the data for MC-SPE/CNCs/Fe₃O₄/NAF/anti-CD63 platform and finally (d) was the signal with MC-SPE/CNCs/Fe₃O₄/NAF/anti-CD63/BSA/CD63 layer. Hence, when the bare MC-SPE electrode was modified with the selected CNCs/Fe₃O₄/NAF nanocomposite, a decline displayed in the EIS diameter. However, after the incubation of the antibody layer, the MC-SPE/CNCs/Fe₃O₄/NAF/anti-CD63/BSA platform shows an increase in the semi-circle diameter due to the formation of an insulating and non-conductive layer. Finally, after the immobilization of antigen and BSA to form MC-SPE/CNCs/Fe₃O₄/NAF/anti-CD63/BSA/CD63 surface, the diameter of the semicircle was further increased which was attributable to the enhanced thickness of the non-conductive layer in curve d of FIG. 12A. Thus, EIS analysis successfully reassured the layer-by-layer characterization of the immunosensor layers.

Next, Cyclic Voltammetry (CV) was performed to analyze the layers on MCSPE/CNCs/Fe₃O₄/NAF/anti-CD63/BSA/CD63 platform. The FIG. 12B represents the signal of the absorbed layers, where (a) bare MC-SPE shows the lowest signal, (b) the highest signal due to highly electrochemical conductive properties of the CNCs/Fe₃O₄/NAF nanocomposite, (c) the MC-SPE/CNCs/Fe₃O₄/NAF/anti-CD63 layer displays a noticeable lower signal than (b) and finally, (d) had the lowest signal representing the SPE/CNCs/Fe₃O₄/NAF/anti-CD63/BSA/CD63 platform, where together BSA and CD63 formed a thick layer on the working electrode surface giving the lowest signal. Moreover, the CV characterization pattern is similar to the signal pattern in ECL, further confirming the successful adsorption of each layer in the fabrication of MC-SPE/CNCs/Fe₃O₄/NAF/antiCD63/BSA/CD63 immunosensor.

Hence, the MC-SPE/CNCs/Fe₃O₄/NAF/antiCD63/BSA immunosensor has a wide linear range of 100 fg mL⁻¹ to 10 ng mL⁻¹ to detect CD63. Both CNCs and Fe₃O₄ provide highly accelerate ECL intensity over MC-SPE in optimal conditions. The fabricated immunosensor has good reproducibility and specificity in real-time. The immunosensor can detect other clinically important proteins including such as but not limited to CD69, and CD81.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. The description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Numerous embodiments of the invention are possible. The previous exemplary embodiments are intended to merely illustrate, and not limit, the breadth and depth of embodiments that can fall within the scope of the appended claims and future claims, which define the invention. For example, the apparatus will be scaled to accommodate different flow rates of the water to be treated or impregnated. The chemical flow rates, hence the concentration of the chemistry, and the pressure in the system may be adjusted depending on the contaminants to be treated and/or the particular application.

It will be apparent that various other modifications and adaptations of the application will be apparent to the person skilled in the art after reading the foregoing disclosure without departing from the spirit and scope of the application and it is intended that all such modifications and adaptations come within the scope of the appended claims.

What is claimed is:

1. A nanocomposite comprising:
carbon nanochips (CNCs);
iron oxide (Fe₃O₄); and
nafion (NAF).

2. The nanocomposite of claim 1, further comprising:
at least 10 μg mL⁻¹ of the CNCs;
at least 20 μg mL⁻¹ of the Fe₃O₄, and
at least 0.25% of the NAF in 1:1 ratio.

3. An electrochemiluminescence immunosensor comprising:
an electrode modified by a nanocomposite comprising a mixture of carbon nanochips (CNCs);
iron oxide (Fe₃O₄); and
nafion (NAF).

4. The electrochemiluminescence immunosensor of claim 3, wherein:
the electrode is a screen-printed electrode.

5. The electrochemiluminescence immunosensor of claim 3, further comprising:
the screen-printed electrode is a carbon screen-printed electrode (SPE).

6. The electrochemiluminescence immunosensor of claim 3, further comprising:
the carbon screen-printed electrode (SPE) is a mesoporous carbon screen-printed electrode (SPE).

7. The electrochemiluminescence immunosensor of claim 3, further comprising:
Ru(bpy)₃Cl₂.6H₂O is a luminophore.

8. The electrochemiluminescence immunosensor of claim 3, further comprising:
TPrA is a coreactant of the luminophore.

9. The electrochemiluminescence immunosensor claim 3, further comprising:
[Ru(bpy)₃]²⁺/TPrA complex formation between Ru(bpy)₃Cl₂.6H₂O and TPrA

10. The electrochemiluminescence immunosensor of claim 3, further comprising:
an electrostatic interaction between the nanocomposite and positively charged luminophore over the MC-SPE/CNCs/Fe₃O₄/NAF electrode.

11. The electrochemiluminescence immunosensor of claim 3, further comprising:
an electron transfer between the modified-electrode surface (MC-SPE/CNCs/Fe₃O₄/NAF) and [Ru(bpy)₃]²⁺/TPrA complex via redox reaction.

12. The electrochemiluminescence immunosensor of claim 3, further comprising:
the nanocomposite comprising at least 10 μg mL⁻¹ of the CNCs; and at least 20 μg mL⁻¹ of the Fe₃O₄, and at least 0.25% of the NAF in 1:1 ratio.

13. The electrochemiluminescence immunosensor of claim 3, wherein
the immunosensor has a protein detection range of 100 fg mL⁻¹ to 10 ng mL⁻¹.

14. The electrochemiluminescence immunosensor claim 3, wherein
the protein is selected from one or more of CD63, CD69, and CD81.

15. A method for fabricating an electrochemiluminescence (ECL) immunosensor detecting target proteins, the ECL immunosensor comprising an electrode modified by a nanocomposite comprising a mixture of carbon nanochips (CNCs); iron oxide (Fe₃O₄); and nafion (NAF), the method comprising:
dropping the nanocomposite over bare MC-SPF and drying thereof for at least 2 hours to form CNC/Fe₃O₄/NAF nanocomposite modified working electrode;
spiking anti-target solution over modified-electrode/CNC/Fe₃O₄/NAF;
incubating the solution overnight at 4° C. to immobilize onto the modified-electrode surface by chemisorption;
washing modified-electrode/CNC/Fe₃O₄/NAF/anti-target using phosphate-buffered saline (PBS) to remove loosely bound antibody and drying thereof at room temperature (RT);
spiking BSA over modified-electrode/CNC/Fe₃O₄/NAF/anti-target as a blocking agent to minimize the non-specific binding and leading to formation of modified-electrode/CNC/Fe₃O₄/NAF/anti-target/BSA; and
washing the modified-electrode/CNC/Fe₃O₄/NAF/anti-target/BSA using PBS and drying thereof at RT, fabricating the modified-electrode/CNC/Fe₃O₄/NAF/anti-target/BSA nanoimmunosensor and storing thereof.

16. The method of claim 15, wherein:
the electrode is a carbon screen-printed electrode.

17. The method of claim 16, further comprising:
the screen-printed electrode is a carbon screen-printed electrode (SPE).

18. The method of claim 17, further comprising:
the carbon screen-printed electrode (SPE) is a mesoporous carbon screen-printed electrode (SPE).

19. The method of claim 18, further comprising:
generating light by [Ru(bpy)₃]²⁺/TPrA complex to detect anti-CD63 over the fabricated nanoimmunosensor.

20. The method of claim 15, wherein
the target protein selected from one or more of CD63, CD69, and CD81.
